(19) United States
(12) Patent Application Publication
(10) Pub. No.: US 2026/0034190 A1
(43) Pub. Date: Feb. 5, 2026

(54) METHOD FOR PRECISELY CONTROLLING FRACTIONAL EXTRACTION OF BIOACTIVE SUBSTANCES OF KAEMPFERIA ELEGANS BY UTILIZING HIGH-VOLTAGE PULSED ELECTRIC FIELD

(71) Applicant: SOUTH CHINA UNIVERSITY OF TECHNOLOGY, Guangzhou (CN)

(72) Inventors: Zhong HAN, Guangzhou (CN); Huanqing LEI, Guangzhou (CN); Xinan ZENG, Guangzhou (CN); Jinlin CAI, Guangzhou (CN)

(73) Assignee: SOUTH CHINA UNIVERSITY OF TECHNOLOGY, Guangzhou (CN)

(21) Appl. No.: 18/995,858

(22) PCT Filed: Oct. 27, 2023
(86) PCT No.: PCT/CN2023/127025

§ 371 Date: Jan. 17, 2025

(30) Foreign Application Priority Data
Nov. 21, 2022 (CN) 202211473176.X

(51) Int. Cl. A61K 36/906

(52) U.S. Cl. CPC A61K 36/906; A61K 2236/13; A61K 2236/15; A61K 2236/17; A61K 2236/333; A61K 2236/51; A61K 2236/53

(57) ABSTRACT

A method for precisely controlling fractional extraction of bioactive substances of Kaempferia elegans by utilizing a high-voltage pulsed electric field is provided. By adjusting the magnitude of energy of the pulsed electric field to precisely control the pore size range of plant cell walls, fractional extraction of polyphenolic flavones, essential oil and polysaccharides in Kaempferia elegans is achieved, the bioactive substances of Kaempferia elegans with high yield, high purity and high activity are efficiently obtained, a large amount of separation cost is reduced, and full utilization of the bioactive substances of Kaempferia elegans is achieved. The extracted bioactive substances can be used for developing functional foods, health care products and the like, can also be applied to the fields of medicine, clinical treatment and the like, and have wide application prospects.

### CROSS REFERENCE TO THE RELATED APPLICATIONS

This application is the national phase entry of International Application No. PCT/CN2023/127025, filed on Oct. 27, 2023, which is based upon and claims priority to Chinese Patent Application No. 202211473176.X, filed on Nov. 21, 2022, the entire contents of which are incorporated herein by reference.

### TECHNICAL FIELD

The present invention relates to a method for precisely controlling fractional extraction of bioactive substances of Kaempferia elegans by utilizing a high-voltage pulsed electric field.

### BACKGROUND

Plants of the genus Kaempferia in Zingiberaceae, represented by rhizoma Kaempferiae as a medicinal and edible homologous plant, are characteristic plants in Guangdong Province. At present, many domestic and foreign experts and scholars have studied chemical components and nutritional values of rhizoma Kaempferiae, and have found that roots and stems of rhizoma Kaempferiae mainly include the chemical components of diterpenes, diphenylheptanes, simple aromatic hydrocarbons, phenylpropanoids, fatty acid esters, flavonoids, phenolic acids, etc. Among the components, polyphenolic flavone substances have oxidation resistance and other activities, and essential oil has the effects of sterilization and pest suppression. In addition, rhizoma Kaempferiae has a relatively high nutritional value and contains large amounts of carbohydrates and proteins and a small amount of fat. Among the plants of the genus Kaempferia in Zingiberaceae, the variety with lilac flowers has a scientific name of Kaempferia elegans (Wall.) Bak., whose roots and stems have a fragrant smell and a spicy taste and contain abundant polyphenolic flavonoid substances, essential oil and active polysaccharides. Moreover, the variety is simple and quick to cultivate and breed and has a potential development value.

At present, polyphenolic flavonoid bioactive substances of plants are usually extracted by utilizing water extraction or organic solvent extraction, supercritical fluid extraction, etc. However, all of these methods have certain defects. The water extraction or organic solvent extraction has damage to activities at high temperature and has low efficiency. The supercritical fluid extraction is expensive in price and is not suitable for industrial production. Essential oil is usually extracted by utilizing a steam distillation method, a solvent extraction method, a supercritical extraction method, etc. The steam distillation method has a long time. A solvent used in the solvent extraction method is easily flammable and explosive and is thus more dangerous. The supercritical extraction method cannot achieve large-scale production due to a high cost. Plant polysaccharides are usually extracted by utilizing a hot water extraction method, an acid-base extraction, an enzymatic method, etc. However, the hot water extraction method has long time consumption and low extraction efficiency. The acid-base extraction method is prone to destroying structures of polysaccharide fragments. The enzymatic method is relatively expensive. All of the methods are not suitable for industrial large-scale production.

At present, the research on extracting active substances of plants by utilizing a pulsed electric field is limited to using effects of the pulsed electric field to destroy cell walls and cell membranes, and then extracting single active substances by combining with ultrasonic extraction, hot water extraction, solvent extraction and other methods. For example, CN103665177B discloses a method for extracting polysaccharides of Portulaca oleracea by utilizing a high-voltage pulsed electric field. CN113354749A discloses a method for extracting water-soluble soybean polysaccharides in soybean residues by utilizing a high-voltage pulsed electric field in collaboration with ultrasonic extraction. Although the extraction efficiency of active substances is improved by the above methods, precise control of extraction is not achieved, a variety of active substances contained in plants are not fully developed and utilized, and further improvements are required.

### SUMMARY

### Technical Issues

The purpose of the present invention is to provide a method for precisely controlling fractional extraction of bioactive substances of Kaempferia elegans by utilizing a high-voltage pulsed electric field. By adjusting the magnitude of energy of the pulsed electric field to precisely control the pore size range of plant cell walls, fractional extraction of polyphenolic flavones, essential oil and polysaccharides in Kaempferia elegans is sequentially achieved, the bioactive substances with high yield, high purity and high activity are efficiently obtained and have wide application prospects, and the problems that precise control of extraction is not achieved in the prior art and a variety of active substances contained in plants are not fully developed and utilized are solved.

### Technical Solutions

The present invention is achieved through the following technical solution:
a method for precisely controlling fractional extraction of bioactive substances of Kaempferia elegans by utilizing a high-voltage pulsed electric field, which includes the following steps:
   (1) after picking Kaempferia elegans plants, selecting their roots and stems, washing the roots and stems, placing the same in a hot air drying oven for drying at 40-45° C. overnight, and then performing crushing and sifting to obtain a Kaempferia elegans powder;
   (2) after adding the Kaempferia elegans powder and an ethanol aqueous solution according to a material-to-liquid ratio of 1:10 to 1:40 for uniform stirring, connecting a peristaltic pump, adjusting a flow rate to 5-20 L/h and removing air bubbles to enable a sample to fully flow through a treatment chamber of a pulsed electric field for treatment by the pulsed electric field, then performing centrifugation, and subjecting a resulting supernatant to freeze-drying to obtain polyphenolic flavonoid bioactive substances; wherein conditions for treatment by the pulsed electric field include that: a field intensity is 1-8 kV/cm, a frequency is 100-1,000 Hz, and a pulse number is 10-60;
   (3) after adding water into a precipitate obtained by the centrifugation in step (2) according to a material-to-liquid ratio of 1:10 to 1:40 for uniform stirring, connecting the peristaltic pump, adjusting the flow rate and removing air bubbles to enable a sample to fully flow through the treatment chamber of the pulsed electric field for treatment by the pulsed electric field, subjecting the treated sample to steam distillation for 3-5 h, and performing separation by an oil-water separator to obtain a Kaempferia elegans essential oil; wherein conditions for treatment by the pulsed electric field include that: a field intensity is 20-40 kV/cm, a frequency is 100-1,000 Hz, and a pulse number is 10-60;
   (4) subjecting a material liquid remaining in step (3) to centrifugation to obtain a supernatant, adding anhydrous ethanol with 3-5 times the volume for alcohol precipitation at 3-5° C. for 24 h, performing centrifugation at 5,000 r/min for 15-25 min, selecting and drying a precipitate, and adding an appropriate amount of deionized water for re-dissolution to obtain a polysaccharide solution; and
   (5) after adding a Sevage reagent with ¼ to ⅓ of the volume of the polysaccharide solution into the polysaccharide solution obtained in step (4) for stirring at room temperature for 15-30 min, performing centrifugation at 5,000 r/min for 8-15 min to remove an upper supernatant and an intermediate protein phase, selecting a lower aqueous phase, adding a Sevage reagent with ¼ to ⅓ of the volume of the lower aqueous phase, and repeating the above step of removing the upper supernatant and the intermediate protein phase for 5 times or above until no denatured proteins are obtained in the intermediate layer; and subjecting a resulting lower aqueous phase solution to freeze-drying to obtain polysaccharides.

It needs to ensure stable flow of an emulsion at the flow rate of 5-20 L/h during the treatment in steps (2) and (3).

Preferably, the material-to-liquid ratio in step (2) is 1:30 to 1:40.

Preferably, a mass fraction of the ethanol aqueous solution in step (2) is 40-50 wt %.

Preferably, the material-to-liquid ratio in step (3) is 1:30 to 1:40.

Preferably, the conditions for treatment by the pulsed electric field in step (2) include that: the field intensity is 5-8 kV/cm, the frequency is 500-1,000 Hz, and the pulse number is 30-60.

Preferably, the conditions for treatment by the pulsed electric field in step (3) include that: the field intensity is 30-40 kV/cm, the frequency is 500-1,000 Hz, and the pulse number is 30-60.

According to the present invention, by adjusting the magnitude of energy of the pulsed electric field to precisely control the formation of small holes with different pore sizes in cell walls, the small holes at 5-10 nm are firstly formed at the low field intensity of 1-8 kV/cm. Under the action of the pulsed electric field and free radicals, the polyphenolic flavonoid substances directionally flow to the outsides of cells through the small holes of the cell walls along transport channels of the plant cells. The treatment by the pulsed electric field is treatment by a low-temperature physical field, such that the activity of the polyphenolic flavonoid substances can be better retained. Then, the small holes at 40-60 nm are formed in the cell walls at the high field intensity of 20-40 kV/cm, thereby improving the essential oil extraction efficiency of a steam distillation method and obtaining the essential oil with high purity. After the above precise fractional extraction is completed, the remaining material liquid is used for extracting the polysaccharides with high activity and high purity. By using the method capable of effectively and precisely controlling the pore size range of plant cell walls, fractional extraction of polyphenolic flavones, essential oil and polysaccharides in Kaempferia elegans is achieved, the bioactive substances with high yield, high purity and high activity are efficiently obtained, a large amount of separation cost after extraction of the bioactive substances is reduced, and full utilization of the bioactive substances of Kaempferia elegans is achieved. The extracted bioactive substances can be used for developing functional foods, health care products and the like, can also be applied to the fields of medicine, clinical treatment and the like, and have wide application prospects.

### Beneficial Effects

The present invention has the following beneficial effects.
1) According to the present invention, by adjusting the magnitude of energy of the pulsed electric field to precisely control the pore size range of plant cell walls, fractional extraction of polyphenolic flavones, essential oil and polysaccharides in Kaempferia elegans is achieved, the bioactive substances with high yield, high purity and high activity are efficiently obtained, a large amount of separation cost after extraction of the bioactive substances is reduced, and full utilization of the bioactive substances of Kaempferia elegans is achieved.
2) According to the present invention, Kaempferia elegans that is simple and quick to cultivate and breed and low in cost is used as a raw material for precise regulation and fractional extraction of the bioactive substances, which is of great significance for improving added values of products and expanding economic benefits.
3) The polyphenolic flavonoid substances and volatile essential oil of Kaempferia elegans extracted in the present invention have good antioxidant activity, antibacterial activity and other characteristics, and the polysaccharides of Kaempferia elegans can well inhibit the activity of a-glucosidase, have the potential ability to control blood glucose, have great medicinal values and can also be used for development of functional foods and cosmetics.

### DETAILED DESCRIPTION OF THE EMBODIMENTS

The following is a further description of the present invention and is not a limitation of the present invention.

### Embodiment 1

(1) After Kaempferia elegans plants were picked, roots and stems were selected, washed and placed in a hot air drying oven for drying at 40° C. overnight, and the dried raw materials were crushed and sifted by a universal high-speed crusher to obtain a Kaempferia elegans powder. After the Kaempferia elegans powder and a 40 wt % ethanol aqueous solution were added according to a material-to-liquid ratio of 1:10 for uniform stirring, a peristaltic pump was connected, a flow rate was adjusted to 20 L/h, and air bubbles were removed to enable a sample to fully flow through a treatment chamber of a pulsed electric field for treatment by the pulsed electric field under the conditions that a field intensity was 1 kV/cm, a frequency was 100 Hz, and a pulse number was 10. Centrifugation was performed to obtain a supernatant, and freeze-drying was performed to obtain polyphenolic flavonoid bioactive substances. The absorbance was tested, the content of the polyphenolic flavonoid substances calculated according to a standard curve was 5.8 mg/g, and the purity was 87%.
(2) After water was added into a precipitate obtained by the centrifugation in step (1) according to a material-to-liquid ratio of 1:10 for uniform stirring, the peristaltic pump was connected, the flow rate was adjusted to 20 L/h, and air bubbles were removed to enable a sample to fully flow through the treatment chamber of the pulsed electric field for treatment by the pulsed electric field under the conditions that a field intensity was 20 kV/cm, a frequency was 100 Hz, and a pulse number was 10. The treated sample was subjected to steam distillation for 3 h and then separated by an oil-water separator to obtain a Kaempferia elegans essential oil. The yield of the essential oil was 20 mg/g.
(3) A material liquid remaining in the above step was subjected to centrifugation to obtain a supernatant, anhydrous ethanol with 4 times the volume was added for alcohol precipitation at 4° C. for 24 h, then centrifugation was performed at 5,000 r/min for 20 min, a precipitate was selected and dried, and an appropriate amount of deionized water was added for re-dissolution to obtain a polysaccharide solution.
(4) After a Sevage reagent with ¼ of the volume of the polysaccharide solution was added into the polysaccharide solution obtained in step (3) and stirred at room temperature for 20 min, centrifugation was performed at 5,000 r/min for 10 min to remove an upper supernatant and an intermediate protein phase, a lower aqueous phase was selected, a Sevage reagent with ¼ of the volume of the lower aqueous phase was added, and the above step of removing the upper supernatant and the intermediate protein phase was repeated for 5 times or above until no denatured proteins were obtained in the intermediate layer; and a resulting lower aqueous phase solution was subjected to freeze-drying to obtain polysaccharides. The content of the polysaccharides in an extraction solution was measured by a phenol-sulfuric acid method, respectively. The yield of the polysaccharides was 306 mg/g, and the purity was 88%.

### Comparative Example 1

Referring to Embodiment 1, differences are that the conditions for treatment by the pulsed electric field in step (1) were the same as those in step (2), including that: the field intensity was 20 kV/cm, the frequency was 100 Hz, and the pulse number was 10. The absorbance was tested, and the content of polyphenolic flavonoid substances calculated according to a standard curve was 7.6 mg/g. Because broken holes in cell walls were relatively large at the relatively large field intensity, many impurities flowed out, thereby affecting the purity of polyphenolic flavonoids. The purity was only 58%, thus requiring subsequent complicated separation steps.

### Comparative Example 2:

Referring to Embodiment 1, differences are that the conditions for treatment by the pulsed electric field in step (1) included that: the field intensity was 0.5 kV/cm, the frequency was 100 Hz, and the pulse number was 10. The absorbance was tested, and the content of polyphenolic flavonoid substances calculated according to a standard curve was 0.3 mg/g. Because the field intensity was too small, holes capable of enabling the polyphenolic flavonoid substances to flow out were not formed in cell walls.

### Embodiment 2

(1) After Kaempferia elegans plants were picked, roots and stems were selected, washed and placed in a hot air drying oven for drying at 43° C. overnight, and the dried raw materials were crushed and sifted by a universal high-speed crusher to obtain a Kaempferia elegans powder. After the Kaempferia elegans powder and a 45 wt % ethanol aqueous solution were added according to a material-to-liquid ratio of 1:30 for uniform stirring, a peristaltic pump was connected, a flow rate was adjusted to 10 L/h, and air bubbles were removed to enable a sample to fully flow through a treatment chamber of a pulsed electric field for treatment by the pulsed electric field under the conditions that a field intensity was 5 kV/cm, a frequency was 1,000 Hz, and a pulse number was 30. Centrifugation was performed to obtain a supernatant, and freeze-drying was performed to obtain polyphenolic flavonoid bioactive substances. The absorbance was tested, the content of the polyphenolic flavonoid substances calculated according to a standard curve was 6.9 mg/g, and the purity was 88%.
(2) After water was added into a precipitate obtained by the centrifugation in step (1) according to a material-to-liquid ratio of 1:30 for uniform stirring, the peristaltic pump was connected, the flow rate was adjusted to 10 L/h, and air bubbles were removed to enable a sample to fully flow through the treatment chamber of the pulsed electric field for treatment by the pulsed electric field under the conditions that a field intensity was 20 kV/cm, a frequency was 100 Hz, and a pulse number was 30. The treated sample was subjected to steam distillation for 4 h and then separated by an oil-water separator to obtain a Kaempferia elegans essential oil. The yield of the essential oil was 27 mg/g.
(3) A material liquid remaining in the above step was subjected to centrifugation to obtain a supernatant, anhydrous ethanol with 4 times the volume was added for alcohol precipitation at 4° C. for 24 h, centrifugation was performed at 5,000 r/min for 20 min, a precipitate was selected and dried, and an appropriate amount of deionized water was added for re-dissolution to obtain a polysaccharide solution.
(4) After a Sevage reagent with ¼ of the volume of the polysaccharide solution was added into the polysaccharide solution obtained in step (3) and stirred at room temperature for 20 min, centrifugation was performed at 5,000 r/min for 10 min to remove an upper supernatant and an intermediate protein phase, a lower aqueous phase was selected, a Sevage reagent with ¼ of the volume of the lower aqueous phase was added, and the above step of removing the upper supernatant and the intermediate protein phase was repeated for 5 times or above until no denatured proteins were obtained in the intermediate layer; and a resulting lower aqueous phase solution was subjected to freeze-drying to obtain polysaccharides. The content of the polysaccharides in an extraction solution was measured by a phenol-sulfuric acid method, respectively. The yield of the polysaccharides was 368 mg/g, and the purity was 90%.

### Embodiment 3

(1) After Kaempferia elegans plants were picked, roots and stems were selected, washed and placed in a hot air drying oven for drying at 45° C. overnight, and the dried raw materials were crushed and sifted by a universal high-speed crusher to obtain a Kaempferia elegans powder. After the Kaempferia elegans powder and a 50 wt % ethanol aqueous solution were added according to a material-to-liquid ratio of 1:40 for uniform stirring, a peristaltic pump was connected, a flow rate was adjusted to 5 L/h, and air bubbles were removed to enable a sample to fully flow through a treatment chamber of a pulsed electric field for treatment by the pulsed electric field under the conditions that a field intensity was 8 kV/cm, a frequency was 1,000 Hz, and a pulse number was 60. Centrifugation was performed to obtain a supernatant, and freeze-drying was performed to obtain polyphenolic flavonoid bioactive substances. The absorbance was tested, the content of the polyphenolic flavonoid substances calculated according to a standard curve was 8.1 mg/g, and the purity was 91%.
(2) After water was added into a precipitate obtained by the centrifugation in step (1) according to a material-to-liquid ratio of 1:40 for uniform stirring, the peristaltic pump was connected, the flow rate was adjusted to 5 L/h, and air bubbles were removed to enable a sample to fully flow through the treatment chamber of the pulsed electric field for treatment by the pulsed electric field under the conditions that a field intensity was 40 kV/cm, a frequency was 1,000 Hz, and a pulse number was 60. The treated sample was subjected to steam distillation for 5 h and then separated by an oil-water separator to obtain a Kaempferia elegans essential oil. The yield of the essential oil was 35 mg/g.
(3) A material liquid remaining in the above step was subjected to centrifugation to obtain a supernatant, anhydrous ethanol with 4 times the volume was added for alcohol precipitation at 4° C. for 24 h, centrifugation was performed at 5,000 r/min for 20 min, a precipitate was selected and dried, and an appropriate amount of deionized water was added for re-dissolution to obtain a polysaccharide solution.
(4) After a Sevage reagent with ¼ of the volume of the polysaccharide solution was added into the polysaccharide solution obtained in step (3) and stirred at room temperature for 20 min, centrifugation was performed at 5,000 r/min for 10 min to remove an upper supernatant and an intermediate protein phase, a lower aqueous phase was selected, a Sevage reagent with ¼ of the volume of the lower aqueous phase was added, and the above step of removing the upper supernatant and the intermediate protein phase was repeated for 5 times or above until no denatured proteins were obtained in the intermediate layer; and a resulting lower aqueous phase solution was subjected to freeze-drying to obtain polysaccharides. The content of the polysaccharides in an extraction solution was measured by a phenol-sulfuric acid method, respectively. The yield of the polysaccharides was 403 mg/g, and the purity was 93%.

The above embodiments are preferred embodiments of the present invention, but the embodiments of the present invention are not limited to said embodiments. Any other changes, modifications, substitutions, combinations and simplifications that are made without deviating from the spirit, essence and principles of the present invention shall be equivalent replacement embodiments, which shall be all included within the scope of protection of the present invention.

What is claimed is:

1. A method for precisely controlling a fractional extraction of bioactive substances of Kaempferia elegans by utilizing a high-voltage pulsed electric field, comprising the following steps:
(1) after picking Kaempferia elegans plants, selecting roots and stems, washing the roots and the stems, placing washed roots and washed stems in a hot air drying oven for drying at 40-45° C. overnight, and then performing crushing and sifting to obtain a Kaempferia elegans powder, wherein the hot air drying oven has a temperature of 40-45° C.;
(2) after adding the Kaempferia elegans powder and an ethanol aqueous solution according to a material-to-liquid ratio of 1:10 to 1:40 for uniform stirring, connecting a peristaltic pump, adjusting a flow rate and removing air bubbles to enable a sample to fully flow through a treatment chamber of a pulsed electric field for a treatment by the high-voltage pulsed electric field, then performing a centrifugation, and subjecting a resulting supernatant to freeze-drying to obtain polyphenolic flavonoid bioactive substances; wherein conditions for the treatment by the high-voltage pulsed electric field comprise that: a field intensity is 1-8 kV/cm, a frequency is 100-1,000 Hz, and a pulse number is 10-60;
(3) after adding water into a precipitate obtained by the centrifugation in the step (2) according to a material-to-liquid ratio of 1:10 to 1:40 for uniform stirring, connecting the peristaltic pump, adjusting the flow rate and removing the air bubbles to enable a sample to fully flow through the treatment chamber of the high-voltage pulsed electric field for a treatment by the high-voltage pulsed electric field, subjecting a treated sample to a steam distillation for 3-5 h, and performing a separation by an oil-water separator to obtain a Kaempferia elegans essential oil; wherein conditions for the treatment by the high-voltage pulsed electric field comprise that: a field intensity is 20-40 kV/cm, a frequency is 100-1,000 Hz, and a pulse number is 10-60;
(4) subjecting a material liquid remaining in the step (3) to a centrifugation to obtain a supernatant, adding anhydrous ethanol with 3-5 times a volume for an alcohol precipitation at 3-5° C. for 24 h, performing the centrifugation at 5,000 r/min for 15-25 min, selecting and drying a precipitate, and adding an appropriate amount of deionized water for a re-dissolution to obtain a polysaccharide solution; and
(5) after adding a Sevage reagent with ¼ to ⅓ of a volume of the polysaccharide solution into the polysaccharide solution obtained in the step (4) for stirring at a room temperature for 15-30 min, performing a centrifugation at 5,000 r/min for 8-15 min to remove an upper supernatant and an intermediate protein phase, selecting a lower aqueous phase, adding the Sevage reagent with ¼ to ⅓ of a volume of the lower aqueous phase, and repeating the above step of removing the upper supernatant and the intermediate protein phase for 5 times or above until no denatured proteins are obtained in an intermediate layer; and
subjecting a resulting lower aqueous phase solution to freeze-drying to obtain polysaccharides.

2. The method according to claim 1, wherein the flow rate in the steps (2) and (3) is adjusted to 5-20 L/h.

3. The method according to claim 1, wherein the material-to-liquid ratio in the step (2) is 1:30 to 1:40.

4. The method according to claim 1, wherein a mass fraction of the ethanol aqueous solution in the step (2) is 40-50 wt %.

5. The method according to claim 1, wherein the material-to-liquid ratio in the step (3) is 1:30 to 1:40.

6. The method according to claim 1, wherein the conditions for the treatment by the high-voltage pulsed electric field in the step (2) comprise that: the field intensity is 5-8 kV/cm, the frequency is 500-1,000 Hz, and the pulse number is 30-60.

7. The method according to claim 1, wherein the conditions for the treatment by the high-voltage pulsed electric field in the step (3) comprise that: the field intensity is 30-40 kV/cm, the frequency is 500-1,000 Hz, and the pulse number is 30-60.
